(19) United States
(12) Patent Application Publication
(10) Pub. No.: US 2024/0293829 A1
(43) Pub. Date: Sep. 5, 2024

(54) METHOD AND APPARATUS FOR BIOLOGICAL CELL HARVESTING

(71) Applicant: Genentech, Inc., South San Francisco, CA (US)

(72) Inventors: Nicholas RUMMEL, South San Francisco, CA (US); Patrick Daniel AHYOW, South San Francisco, CA (US)

(21) Appl. No.: 18/431,838

(22) Filed: Feb. 2, 2024

Related U.S. Application Data
(63) Continuation of application No. PCT/US2022/074500, filed on Aug. 4, 2022.
(60) Provisional application No. 63/260,033, filed on Aug. 6, 2021.

(51) Int. Cl. B04B 7/00; B33Y 80/00; C12M 1/26

(52) U.S. Cl. CPC B04B 7/00; B33Y 80/00; C12M 33/10

(57) ABSTRACT

Provided herein are systems, devices and methods for harvesting biological cells with a centrifuge. A biological cell harvesting centrifuge apparatus may be provided with a rectangularly shaped base and 21 laterally spaced apart upright supports. Each upright support may extend vertically upward from the base and have a hollow core. Each upright support may be configured to slidably receive and support either one, two or four vertical edges of either one, two or four 15 mL micro bioreactor vessels. The 21 upright supports may be arranged in a 3×7 array such that they form a 2×6 array of receptacles configured to slidably receive and support 12 of the bioreactor vessels. The upright supports may be configured to support at least 80% of the vertical edges of the bioreactor vessels.

### CLAIM OF PRIORITY

This application is a continuation of International Application No. PCT/US2022/074500, filed Aug. 4, 2022, which claims the benefit of U.S. Provisional Application No. 63/260,033, filed Aug. 6, 2021, each of which are herein incorporated by reference in its entirety for all purposes.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Biotechnology and pharmaceutical companies often grow cell cultures which are used in biological and medical research, and may also be used commercially in the mass production of therapeutic proteins. Various mammalian cell cultures are useful during drug development, testing and production. For example, Chinese hamster ovary (CHO) cells are an epithelial cell line derived from the ovary of the Chinese hamster. They have found wide use in studies of genetics, toxicity screening, nutrition and gene expression, particularly to express recombinant proteins. CHO cells are often used as mammalian hosts for industrial production of recombinant protein therapeutics.

It is often advantageous to grow a large number of small cell cultures in parallel while varying the parameters of each cell culture. The small cell cultures, often between 10 and 15 mL each, can mimic the characteristics of lab scale bioreactors to enable optimal cell growth, productivity and product quality.

The Ambr® 15 Cell Culture system provided by Sartorius Stedim Biotech of Goettingen, Germany, has become an industry standard automated microbioreactor system for mammalian cell culture. It has applications throughout the industry, most commonly for cell line screening and media/feed development. On each Ambr® 15 workstation, conditions in up to 48×15 mL bioreactors can be individually controlled while a liquid handler enables automated addition and removal of liquids during the process. Integrated cell counting, metabolite analysis and pH offset correction are also possible, thereby reducing the operator interactions that are required.

While the Ambr® 15 Cell Culture system has been in use for many years, processes that utilize the system continue to evolve. Accordingly, what is needed and is not provided by the prior art is improved devices, systems and methods for making existing processes more efficient, thereby reducing costs and increasing the speed of biological and medical research. The innovations described herein solve these unmet needs and provide additional advantages.

### SUMMARY OF THE DISCLOSURE

According to aspects of the present disclosure, a biological cell harvesting centrifuge apparatus may be provided with a rectangularly shaped base and 21 laterally spaced apart upright supports. In some embodiments, each upright support extends vertically upward from the base. Each upright support may have a hollow core and may be configured to slidably receive and support either one, two or four vertical edges of either one, two or four 15 mL micro bioreactor vessels. In some embodiments, the 21 upright supports are arranged in a 3×7 array such that they form a 2×6 array of receptacles configured to slidably receive and support 12 of the bioreactor vessels. The upright supports may be configured to support at least 80% of the vertical edges of the bioreactor vessels.

In some embodiments, the apparatus has a unitary construction comprising a single piece of material. The apparatus may be formed of a thermoplastic and or may be formed by a 3D printing process.

In some embodiments, each upright support has a top end and a bottom end and the base of the apparatus includes 32 horizontal support segments. Each of the horizontal support segments may span between the bottom ends of two upright supports. In some embodiments, each of the horizontal support segments receives and supports either one or two horizontal bottom edges of either one or two of the bioreactor vessels such that the apparatus supports 8 edges of each of the 12 bioreactor vessels it receives. Each of the horizontal support segments may include a hollow core. In some embodiments, the base of the apparatus includes an opening beneath each of the bioreactor receptacles and between the horizontal support segments. In some embodiments, the apparatus contacts only edges of the bioreactor vessels and does not contact central portions of side or bottom faces of the vessels.

In some embodiments, each upright support has a top end and a bottom end and the top ends of at least two pairs of upright supports located on a periphery of the apparatus are each connected with a handle spanning therebetween. The top ends of four pairs of upright supports located adjacent to corners of the apparatus may each be connected with a handle spanning therebetween, and the top ends of the remaining upright supports may not be interconnected. In some embodiments, each of the handles cooperates with the connected upright supports to form an arch beneath the handle.

In some embodiments, the base includes a pair of bosses and each of the bosses extends laterally from opposite sides of the base. Each of the bosses may be configured to contact an inner surface of a centrifuge receptacle such that the apparatus is prevented from moving laterally within the centrifuge receptacle during use. In some embodiments, each of the bosses spans between at least three upright supports. In some embodiments, the base includes two pair of bosses with each of the pairs of bosses extending laterally from opposite sides of the base. Each of the pairs of bosses may be configured to contact an inner surface of a centrifuge receptacle such that the apparatus is prevented from moving laterally within the centrifuge receptacle during use. In some embodiments, each of the bosses is located adjacent to a corner of the base.

In some embodiments, a biological cell harvesting centrifuge apparatus is provided with a rectangularly shaped base and 21 laterally spaced apart upright supports. Each upright support may extend vertically upward from the base and may have a hollow core. In some embodiments, each upright support is configured to slidably receive and support either one, two or four vertical edges of either one, two or four 15 mL micro bioreactor vessels. The 21 upright supports may be arranged in a 3×7 array such that they form a 2×6 array of receptacles configured to slidably receive and support 12 of the bioreactor vessels. In some embodiments, the upright supports are configured to support at least 80% of the vertical edges of the bioreactor vessels. Each upright support may have a top end and a bottom end, and the base of the apparatus may include 32 horizontal support segments, wherein each of the horizontal support segments spans between the bottom ends of two upright supports. In some embodiments, each of the horizontal support segments receives and supports either one or two horizontal bottom edges of either one or two of the bioreactor vessels such that the apparatus supports 8 edges of each of the 12 bioreactor vessels it receives. Each of the horizontal support segments may include a hollow core. In some embodiments, the base of the apparatus includes an opening beneath each of the bioreactor receptacles and between the horizontal support segments. In some embodiments, the apparatus contacts only edges of the bioreactor vessels and does not contact central portions of side or bottom faces of the vessels. In some embodiments, each upright support has a top end and a bottom end and the top ends of four pairs of upright supports located adjacent to corners of the apparatus are each connected with a handle spanning therebetween. In these embodiments, the top ends of the remaining upright supports are not interconnected. Each of the handles may cooperate with the connected upright supports to form an arch beneath the handle. In some embodiments, the base includes a pair of bosses and each of the bosses extends laterally from opposite sides of the base. Each of the bosses may be configured to contact an inner surface of a centrifuge receptacle such that the apparatus is prevented from moving laterally within the centrifuge receptacle during use. In some embodiments, the apparatus has a unitary construction comprising a single piece of plastic.

According to aspects of the present disclosure, a method of harvesting biological cells may include the steps of providing a plurality of 15 mL micro bioreactor vessels and adding biological material to the plurality of vessels. The biological material may then be cultivated within the plurality of vessels. In some embodiments, the plurality of vessels with their biological material remaining therein are inserted into a vessel holding apparatus. The vessel holding apparatus with the plurality of vessels may be placed into a centrifuge. The method may further include spinning the vessel holding apparatus and vessels with the centrifuge and removing the biological material from the vessels to harvest the cells.

In some embodiments of the above method, the vessel holding apparatus includes at least 12 receptacles and each of the receptacles is configured to slidable receive one of the plurality of vessels. The providing step may include providing 24 15 mL micro bioreactor vessels, and the inserting step may include inserting the 24 vessels into two vessel holding apparatuses, each of the vessel holding apparatuses holding 12 vessels. In some embodiments, the placing step includes placing both of the vessel holding apparatuses into the centrifuge, and the spinning step includes spinning the two vessel holding apparatuses and the 24 vessels in the centrifuge simultaneously.

In some embodiments, the providing step includes providing 48 15 mL micro bioreactor vessels and the inserting step includes inserting the 48 vessels into four vessel holding apparatuses. Each of the vessel holding apparatuses may hold 12 vessels. In some embodiments, the placing step includes placing all four of the vessel holding apparatuses into the centrifuge, and the spinning step includes spinning the four vessel holding apparatuses and the 48 vessels in the centrifuge simultaneously.

In some embodiments of the above method, the vessel holding apparatus includes 21 laterally spaced apart upright supports and each upright support extends vertically upward from the base. Each upright support may have a hollow core. In some embodiments, each upright support is configured to slidably receive and support either one, two or four vertical edges of either one, two or four 15 mL micro bioreactor vessels. The 21 upright supports may be arranged in a 3×7 array such that they form a 2×6 array of receptacles configured to slidably receive and support 12 of the bioreactor vessels. In some embodiments, the upright supports are configured to support at least 80% of the vertical edges of the bioreactor vessels.

In some embodiments, each upright support of the vessel holding apparatus has a top end and a bottom end and the base of the apparatus includes 32 horizontal support segments. Each of the horizontal support segments may span between the bottom ends of two upright supports. In some embodiments, each of the horizontal support segments receives and supports either one or two horizontal bottom edges of either one or two of the bioreactor vessels such that the apparatus supports 8 edges of each of the 12 bioreactor vessels it receives.

In some embodiments, the vessel holding apparatus includes a rectangularly shaped base and 21 laterally spaced apart upright supports. Each upright support may extend vertically upward from the base and each upright support may have a hollow core. In some embodiments, each upright support is configured to slidably receive and support either one, two or four vertical edges of either one, two or four 15 mL micro bioreactor vessels. The 21 upright supports may be arranged in a 3×7 array such that they form a 2×6 array of receptacles configured to slidably receive and support 12 of the bioreactor vessels. In some embodiments, the upright supports are configured to support at least 80% of the vertical edges of the bioreactor vessels and each upright support has a top end and a bottom end. The base of the apparatus may include 32 horizontal support segments, wherein each of the horizontal support segments spans between the bottom ends of two upright supports. In some embodiments, each of the horizontal support segments receives and supports either one or two horizontal bottom edges of either one or two of the bioreactor vessels such that the apparatus supports 8 edges of each of the 12 bioreactor vessels it receives. Each of the horizontal support segments may include a hollow core. In some embodiments, the base of the apparatus includes an opening beneath each of the bioreactor receptacles and between the horizontal support segments. In some embodiments, the apparatus contacts only edges of the bioreactor vessels and does not contact central portions of side or bottom faces of the vessels. Each upright support may have a top end and a bottom end and the top ends of four pairs of upright supports located adjacent to corners of the apparatus may each be connected with a handle spanning therebetween. In these embodiments, the top ends of the remaining upright supports are not interconnected. Each of the handles may cooperate with the connected upright supports to form an arch beneath the handle. In some embodiments, the base includes a pair of bosses, each of the bosses extending laterally from opposite sides of the base. Each of the bosses may be configured to contact an inner surface of a centrifuge receptacle such that the apparatus is prevented from moving laterally within the centrifuge receptacle during use. In some of these embodiments, the apparatus has a unitary construction comprising a single piece of plastic.

### BRIEF DESCRIPTION OF THE DRAWINGS

The novel features of the disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 is a perspective view showing a prior art 15 mL micro bioreactor vessel;

FIG. 2 is a perspective view showing a biological cell harvesting centrifuge apparatus constructed according to aspects of the disclosure;

FIG. 3 is a top plan view showing the apparatus of FIG. 2;

FIG. 4 is side-elevation view showing the apparatus of FIG. 2;

FIG. 5 is an end view showing the apparatus of FIG. 2;

FIG. 6 is a perspective view showing two of the apparatuses of FIG. 2 filled with bioreactor vessels and placed in a centrifuge according to aspects of the disclosure; and

FIG. 7 is an enlarged perspective view showing a portion of one of the apparatuses of FIG. 6 without bioreactor vessels inserted.

### DETAILED DESCRIPTION

As previously described in the Background section of this disclosure, The Ambr® 15 Cell Culture system provided by Sartorius Stedim Biotech of Goettingen, Germany, has become an industry standard automated microbioreactor system for mammalian cell culture. Referring to FIG. 1, a standard 15 mL micro bioreactor vessel 100 is provided for use with the Ambr® 15 system. Forty-eight disposable bioreactors 100 are typically used together at one time. Each bioreactor 100 includes a transparent plastic body 110, a top port 112 for liquid additions and sampling, a stirring impeller 114, a gas sparge tube 116, a pH sensor 118 and a dissolved oxygen sensor 120. Bioreactor 100 has a width of 28.1 mm at the height of the impeller.

In some prior art processes, after sufficient cell growth has occurred, cell culture fluid from each bioreactor 100 is aspirated from the bioreactor and placed into a separate Falcon® tube (not shown.) The 48 Falcon® tubes are then placed in one or more racks, such as a standard, deep 96-well plate (not shown.) The well plate(s) are then placed in a centrifuge and spun to separate the cells that are to be harvested.

According to aspects of the present disclosure, Applicants have discovered that during centrifugation, the cell culture fluid can remain in the bioreactors 100, and the bioreactors themselves can be loaded into the centrifuge and spun without needing to transfer the contents of the bioreactors 100 to Falcon® tubes or other containers first. This, however, requires a special apparatus to securely house the bioreactors during centrifugation. Not transferring the cell culture fluid out of the bioreactors before centrifugation provides a number of advantages, including the following. First, Falcon® tubes, pipettes and or other supplies needed to transfer the fluid are no longer required. Second, significant process time is saved by not transferring the fluid. Third, extra lab technician labor costs, and or the costs associated with utilizing automated equipment to transfer the fluid, are avoided. Fourth, ergonomics are improved by avoiding the highly repetitive actions associated with transferring the contents of many vessels. These and other advantages will be become apparent from the following detailed description of the inventive methods, systems and apparatuses.

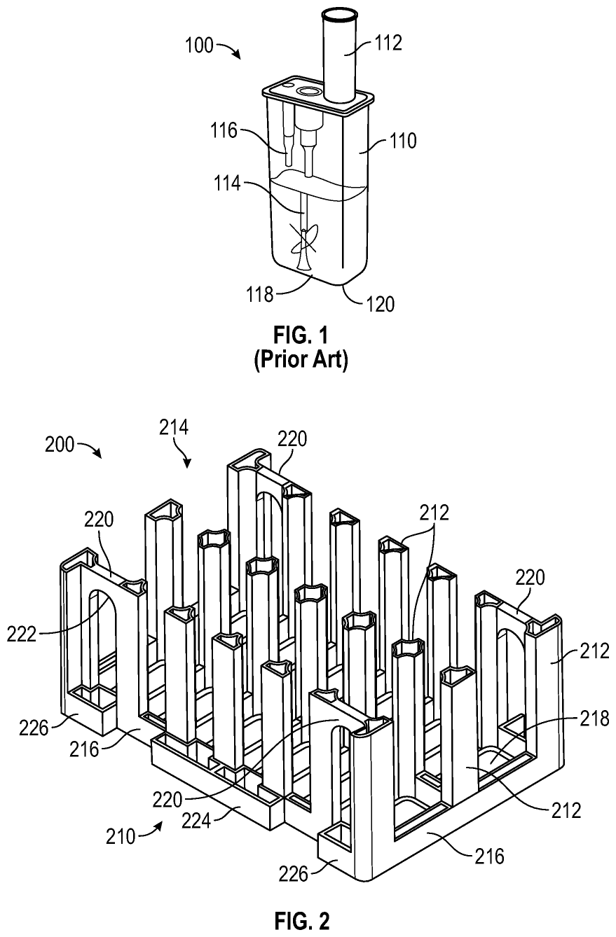

Referring to FIGS. 2-5, an exemplary biological cell harvesting centrifuge apparatus 200 constructed according to aspects of the present disclosure is shown. In this exemplary embodiment, apparatus 200 includes a rectangularly shaped base 210 and 21 laterally spaced apart upright supports 212. Each upright support 212 extends vertically upward from the base and may be integrally formed therewith. Each upright support may have a hollow core as shown. The hollow core arrangement allows apparatus 200 to be constructed more quickly, particularly in embodiments where apparatus 200 is fabricated with an additive manufacturing process such as 3D printing. In embodiments where apparatus 200 is molded from a thermoplastic, the hollow cores of upright supports 212 allow the supports to cool with less shrinking and or warping. In embodiments where apparatus 200 is formed of metal, such as an aluminum alloy, the hollow cores of upright supports 212 may be formed from extruded tubes or a casting process to provide a lighter apparatus for use in a centrifuge.

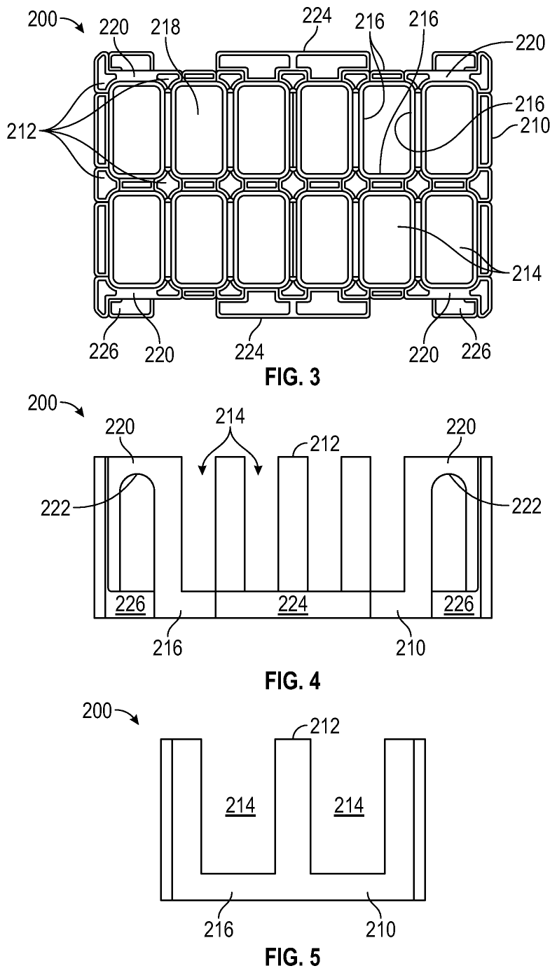

In the exemplary embodiment shown in FIGS. 2-5, upright supports 212 are arranged in a 3×7 array (i.e. arranged in 3 columns, each column having 7 upright supports 212) such that the supports form a 2×6 array of receptacles 214, as best seen in FIG. 3. Each receptacle 214 is configured to slidably receive and support one bioreactor vessel 100 (shown in FIG. 1.) Each upright support 212 is configured to slidably receive and support either one, two or four vertical edges of either one, two or four bioreactor vessels 100. In particular, the four upright supports 212 located near the corners of base 210 may each be configured with an L-shaped cross-section to support one vertical edge of a bioreactor vessel 100. The twelve upright supports 212 located near the periphery of base 210 between the corners may each be configured with a T-shaped cross-section to each support two vertical edges of two adjacent bioreactor vessels 100. The five upright supports 212 located in the central portion of base 210 may each be configured with an X-shaped cross-section to support four vertical edges of four adjacent bioreactor vessels 100. In this exemplary embodiment, bioreactor vessels 100 are held securely in place by upright supports 212 contacting only the edges of the vertical sides of vessels 100. In other words, apparatus 200 does not contact the central portions of the side faces of vessels 100.

In this exemplary embodiment, upright supports 212 are configured to support at least 80% of the vertical edges of bioreactor vessels 100. In this embodiment, 80% of the height of vessels 100 resides within receptacles 214 formed by upright supports 212 and 20% of the height extends above supports 212. In other embodiments (not shown), upright supports 212 are configured to support at least 40%, at least 50%, at least 60%, at least 70%, at least 90% or 100% of the vertical edges of bioreactor vessels 100.

In other embodiments (not shown), upright supports 212 may be arranged in a 5×4 array (i.e. arranged in 5 columns, each column having 4 upright supports 212) such that the supports form a 4×3 array of receptacles 214. Such an arrangement has a similar footprint as that of apparatus 200 for fitting into a compartment of a centrifuge. Depending on the centrifuge configuration, other numbers and or arrangements of receptacles 214 are possible.

As best seen in FIG. 3, base 210 of exemplary apparatus 200 is formed from 32 horizontal support segments 216. Each of the horizontal support segments 216 spans between the bottom ends of two upright supports 212. Each of the horizontal support segments 216 receives and supports either one or two horizontal bottom edges of either one or two of the bioreactor vessels 100. In particular, the 16 horizontal support segments 216 located near the perimeter of base 210 may each be configured with an L-shaped or F-shaped cross-section to support one horizontal edge of a bioreactor vessel 100. The 16 horizontal support segments 216 located in the central portion of base 210 may each be configured with an inverted T-shaped cross-section to each support two horizontal edges of two adjacent bioreactor vessels 100. With this arrangement, an opening 218 is provided beneath each of the bioreactor receptacles 214 and between the horizontal support segments 216. In this exemplary embodiment, bioreactor vessels 100 are held securely in place by horizontal support segments 216 contacting only the edges of the horizontal sides of vessels 100. In other words, apparatus 200 does not contact the central portions of the bottom faces of vessels 100. In combination, upright supports 212 and horizontal support segments 216 cooperate to slidably receive and securely support 8 of the 12 edges of each vessel 100. The top 4 edges of each vessel 100 are not contacted so that vessels 100 may be easily slide into and out of receptacles 214.

In some embodiments, the 16 horizontal support segments 216 located near the perimeter of base 210 may each be configured with an F-shaped cross-section. The concave portion of the F-shaped cross-section may face upward as shown. This arrangement provides strength to the perimeter of apparatus 200 while conveying the same benefits provided by making the upright supports hollow, as previously described. Similarly, the 16 horizontal support segments 216 located in the center portion of base 210 may be provided with downwardly facing recesses (not shown) along their centerlines.

As best seen in FIGS. 2 and 4, a pair of handles 220 may be provided at each end of apparatus 200. Handles 220 may be formed by connecting the top ends of adjacent upright supports 212 located on the periphery of apparatus 200. In some embodiments, each of the handles 220 cooperates with the connected upright supports 212 to form an arch 222 beneath the handle 220, as shown. In this exemplary embodiment, handles 220 are located adjacent to corners of apparatus 200 and the top ends of the remaining upright supports 212 are not interconnected. In other embodiments (not shown), handles 220 may be formed in a mid-portion of one or more sides of apparatus 200, in addition to or instead of handles 220 located at the corners. In some embodiments, one, two, three, four or more pairs of handles may be provided, or the handles may be omitted.

As best seen in FIGS. 2 and 3, a laterally extending boss 224 may be provided on opposite sides of base 210. Each of the two bosses 224 is configured to contact an inner surface of a centrifuge receptacle such that apparatus 200 is prevented from moving laterally within the centrifuge receptacle during use. In this exemplary embodiment, each of the bosses 224 spans between the bottom ends of at least three upright supports 212. In addition to or instead of bosses 224, apparatus 200 may also be provided with two other pairs of bosses 226, such as shown in FIGS. 2 and 3. Each of the pairs of bosses 226 may extend laterally from opposite sides of base 210, each of the pairs of bosses 226 being configured to contact an inner surface of a centrifuge receptacle such that apparatus 200 is prevented from moving laterally within the centrifuge receptacle during use. In this exemplary embodiment, each of the bosses 226 is located adjacent to a corner of base 210.

In this exemplary embodiment, apparatus 200 is 85 mm wide, 127 mm deep and 52 mm tall. In some embodiments, apparatus 200 has a unitary construction comprising a single piece of material. Apparatus 200 may be formed of a thermoplastic, polymer, metal, ceramic or other suitable material. In some embodiments, apparatus 200 is formed by an additive manufacturing process such as 3D printing. In other embodiments, apparatus 200 may be molded, cast, assembled from extruded parts, or fabricated by other suitable processes.

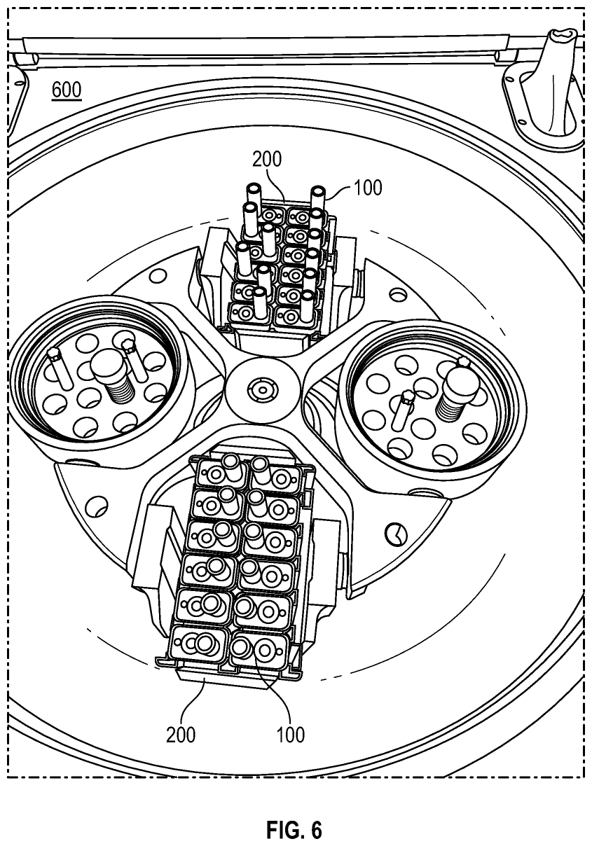

Referring to FIG. 6, two biological cell harvesting centrifuge apparatuses 200 are shown in use, each filled with 12 bioreactor vessels 100 and placed in a centrifuge 600 according to aspects of the present disclosure. In this example, apparatuses 200 are placed in a Sorvall Legend RT centrifuge originally provided by Kendro Laboratory Products of Asheville, North Carolina (now owned by ThermoFisher Scientific of Waltham, Massachusetts.) This centrifuge is provided with a rotor having four receptacles. One, two, three or four apparatuses 200 may be placed in the rotor at a time, with a suitable counterbalance weight placed in any empty receptacles. Other centrifuges may be used, particularly those having receptacles/adapters configured to receive a standard 96-well plate.

In some implementations, cell cultures are grown in 12, 24, 36 or 48 separate bioreactor vessels 100 for 14 days using an Ambr® 15 Cell Culture system (not shown) provided by Sartorius Stedim Biotech of Goettingen, Germany. The bioreactor vessels 100 are then moved from the Ambr® 15 system into one or more harvesting apparatuses 200 (either directly or with a standard bioreactor carrier.) The loaded apparatus(es) 200 may then be placed into the rotor of centrifuge 600. In some implementations, apparatus(es) 200 may each sit in an adapter that fits in one of the four rotor receptacles. In some implementations, centrifuge 600 is then run at 2000 rpm for 10 minutes. Supernatant may then be decanted from each of the centrifuged vessels 100 into labeled 15 mL Falcon® tubes. The used bioreactor vessels 100 with any remaining precipitate inside may be discarded, such as into incineration trash or biohazardous waste receptacles. Apparatus(es) 200 may be repeatedly reused in further centrifuge cell harvesting procedures.

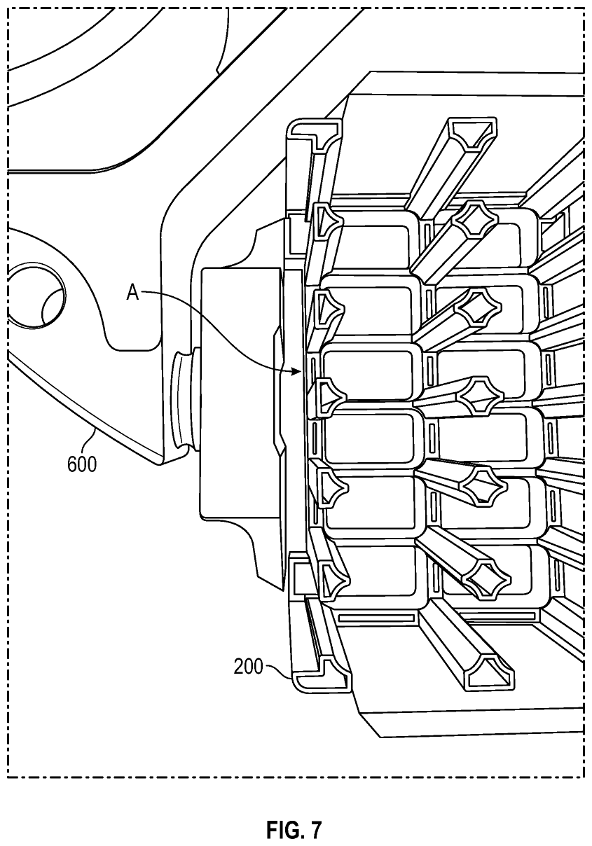

Referring to FIG. 7, an enlarged view of one of the apparatuses 200 in centrifuge 600 is shown. Bioreactor vessels 100 (shown in FIG. 7) and bosses 224 (shown in FIGS. 2-4) have been omitted for clarity. Arrow A shows a gap that can exist between apparatus 200 and the centrifuge rotor receptacle/adapter when bosses 224 are not provided, allowing undesirable movement between apparatus 200 and the rotor of centrifuge 600.

In alternative embodiments (not shown), biological cell harvesting centrifuge apparatuses 200 may be modified to accommodate a different number or configuration of bioreactor vessel. For example, the apparatus may be modified to slidable receive a smaller number of larger vessels, such as 250 mL bioreactor vessels that are about 4 inches in diameter, rather than the 15 mL vessels described above. In some embodiments, the bioreactor vessels may be round or square rather than rectangular, and a combination of sizes and or shapes may be accommodated. In some alternative embodiments, two or more arrays of bioreactor vessels may be stacked on top of one another. The multiple layers of vessels may be interlocked so that they can remain in place during centrifugation even when they may not be directly supported in a centrifuge rotor receptacle. In alternative embodiments, solid divider walls may be utilized instead of discrete upright supports.

While exemplary embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and/or methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. When a feature is described as optional, that does not necessarily mean that other features not described as optional are required.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A biological cell harvesting centrifuge apparatus comprising:
a rectangularly shaped base; and
21 laterally spaced apart upright supports, each upright support extending vertically upward from the base, each upright support having a hollow core, each upright support configured to slidably receive and support either one, two or four vertical edges of either one, two or four 15 mL micro bioreactor vessels, wherein the 21 upright supports are arranged in a 3×7 array such that they form a 2×6 array of receptacles configured to slidably receive and support 12 of the bioreactor vessels, wherein the upright supports are configured to support at least 80% of the vertical edges of the bioreactor vessels.

2. The apparatus of claim 1, wherein the apparatus has a unitary construction comprising a single piece of material.

3. The apparatus of claim 2, wherein the apparatus is formed of a thermoplastic.

4. The apparatus of claim 2, wherein the apparatus is formed by a 3D printing process.

5. The apparatus of claim 1, wherein each upright support has a top end and a bottom end, wherein the base of the apparatus comprises 32 horizontal support segments, wherein each of the horizontal support segments spans between the bottom ends of two upright supports, wherein each of the horizontal support segments receives and supports either one or two horizontal bottom edges of either one or two of the bioreactor vessels such that the apparatus supports 8 edges of each of the 12 bioreactor vessels it receives.

6. The apparatus of claim 5, wherein each of the horizontal support segments comprises a hollow core.

7. The apparatus of claim 5, wherein the base of the apparatus comprises an opening beneath each of the bioreactor receptacles and between the horizontal support segments.

8. The apparatus of claim 5, wherein the apparatus contacts only edges of the bioreactor vessels and does not contact central portions of side or bottom faces of the vessels.

9. The apparatus of claim 1, wherein each upright support has a top end and a bottom end, wherein the top ends of at least two pairs of upright supports located on a periphery of the apparatus are each connected with a handle spanning therebetween.

10. The apparatus of claim 9, wherein the top ends of four pairs of upright supports located adjacent to corners of the apparatus are each connected with a handle spanning therebetween, wherein the top ends of the remaining upright supports are not interconnected.

11. The apparatus of claim 10, wherein each of the handles cooperates with the connected upright supports to form an arch beneath the handle.

12. The apparatus of claim 1, wherein the base comprises a pair of bosses, each of the bosses extending laterally from opposite sides of the base, each of the bosses being configured to contact an inner surface of a centrifuge receptacle such that the apparatus is prevented from moving laterally within the centrifuge receptacle during use.

13. The apparatus of claim 12, wherein each of the bosses spans between at least three upright supports.

14. The apparatus of claim 1, wherein the base comprises two pair of bosses, each of the pairs of bosses extending laterally from opposite sides of the base, each of the pairs of bosses being configured to contact an inner surface of a centrifuge receptacle such that the apparatus is prevented from moving laterally within the centrifuge receptacle during use.

15. The apparatus of claim 14, wherein each of the bosses is located adjacent to a corner of the base.

16. A biological cell harvesting centrifuge apparatus comprising:
a rectangularly shaped base; and
21 laterally spaced apart upright supports, each upright support extending vertically upward from the base, each upright support having a hollow core, each upright support configured to slidably receive and support either one, two or four vertical edges of either one, two or four 15 mL micro bioreactor vessels, wherein the 21 upright supports are arranged in a 3×7 array such that they form a 2×6 array of receptacles configured to slidably receive and support 12 of the bioreactor vessels, wherein the upright supports are configured to support at least 80% of the vertical edges of the bioreactor vessels,
wherein each upright support has a top end and a bottom end, wherein the base of the apparatus comprises 32 horizontal support segments, wherein each of the horizontal support segments spans between the bottom ends of two upright supports, wherein each of the horizontal support segments receives and supports either one or two horizontal bottom edges of either one or two of the bioreactor vessels such that the apparatus supports 8 edges of each of the 12 bioreactor vessels it receives,
wherein each of the horizontal support segments comprises a hollow core,
wherein the base of the apparatus comprises an opening beneath each of the bioreactor receptacles and between the horizontal support segments,
wherein the apparatus contacts only edges of the bioreactor vessels and does not contact central portions of side or bottom faces of the vessels,
wherein each upright support has a top end and a bottom end, wherein the top ends of four pairs of upright supports located adjacent to corners of the apparatus are each connected with a handle spanning therebetween, wherein the top ends of the remaining upright supports are not interconnected,
wherein each of the handles cooperates with the connected upright supports to form an arch beneath the handle,
wherein the base comprises a pair of bosses, each of the bosses extending laterally from opposite sides of the base, each of the bosses being configured to contact an inner surface of a centrifuge receptacle such that the apparatus is prevented from moving laterally within the centrifuge receptacle during use, and
wherein the apparatus has a unitary construction comprising a single piece of plastic.

17. A method of harvesting biological cells, the method comprising:
providing a plurality of 15 mL micro bioreactor vessels;
adding biological material to the plurality of vessels;
cultivating the biological material within the plurality of vessels;
inserting the plurality of vessels with their biological material remaining therein into a vessel holding apparatus;
placing the vessel holding apparatus with the plurality of vessels into a centrifuge;
spinning the vessel holding apparatus and vessels with the centrifuge; and
removing the biological material from the vessels to harvest the cells.

18. The method of claim 17, wherein the vessel holding apparatus comprises at least 12 receptacles, each of the receptacles being configured to slidable receive one of the plurality of vessels.

19. The method of claim 17, wherein the providing step comprises providing 24 15 mL micro bioreactor vessels, wherein the inserting step comprises inserting the 24 vessels into two vessel holding apparatuses, each of the vessel holding apparatuses holding 12 vessels, wherein the placing step comprises placing both of the vessel holding apparatuses into the centrifuge, and wherein the spinning step comprises spinning the two vessel holding apparatuses and the 24 vessels in the centrifuge simultaneously.

20. The method of claim 17, wherein the providing step comprises providing 48 15 mL micro bioreactor vessels, wherein the inserting step comprises inserting the 48 vessels into four vessel holding apparatuses, each of the vessel holding apparatuses holding 12 vessels, wherein the placing step comprises placing all four of the vessel holding apparatuses into the centrifuge, and wherein the spinning step comprises spinning the four vessel holding apparatuses and the 48 vessels in the centrifuge simultaneously.

21. The method of claim 17, wherein the vessel holding apparatus comprises:
a rectangularly shaped base; and
21 laterally spaced apart upright supports, each upright support extending vertically upward from the base, each upright support having a hollow core, each upright support configured to slidably receive and support either one, two or four vertical edges of either one, two or four 15 mL micro bioreactor vessels, wherein the 21 upright supports are arranged in a 3×7 array such that they form a 2×6 array of receptacles configured to slidably receive and support 12 of the bioreactor vessels, wherein the upright supports are configured to support at least 80% of the vertical edges of the bioreactor vessels.

22. The method of claim 21, wherein each upright support of the vessel holding apparatus has a top end and a bottom end, wherein the base of the apparatus comprises 32 horizontal support segments, wherein each of the horizontal support segments spans between the bottom ends of two upright supports, wherein each of the horizontal support segments receives and supports either one or two horizontal bottom edges of either one or two of the bioreactor vessels such that the apparatus supports 8 edges of each of the 12 bioreactor vessels it receives.

23. The method of claim 17, wherein the vessel holding apparatus comprises:
a rectangularly shaped base; and
21 laterally spaced apart upright supports, each upright support extending vertically upward from the base, each upright support having a hollow core, each upright support configured to slidably receive and support either one, two or four vertical edges of either one, two or four 15 mL micro bioreactor vessels, wherein the 21 upright supports are arranged in a 3×7 array such that they form a 2×6 array of receptacles configured to slidably receive and support 12 of the bioreactor vessels, wherein the upright supports are configured to support at least 80% of the vertical edges of the bioreactor vessels,
wherein each upright support has a top end and a bottom end, wherein the base of the apparatus comprises 32 horizontal support segments, wherein each of the horizontal support segments spans between the bottom ends of two upright supports, wherein each of the horizontal support segments receives and supports either one or two horizontal bottom edges of either one or two of the bioreactor vessels such that the apparatus supports 8 edges of each of the 12 bioreactor vessels it receives,
wherein each of the horizontal support segments comprises a hollow core,
wherein the base of the apparatus comprises an opening beneath each of the bioreactor receptacles and between the horizontal support segments,
wherein the apparatus contacts only edges of the bioreactor vessels and does not contact central portions of side or bottom faces of the vessels,
wherein each upright support has a top end and a bottom end, wherein the top ends of four pairs of upright supports located adjacent to corners of the apparatus are each connected with a handle spanning therebetween, wherein the top ends of the remaining upright supports are not interconnected,
wherein each of the handles cooperates with the connected upright supports to form an arch beneath the handle,
wherein the base comprises a pair of bosses, each of the bosses extending laterally from opposite sides of the base, each of the bosses being configured to contact an inner surface of a centrifuge receptacle such that the apparatus is prevented from moving laterally within the centrifuge receptacle during use, and
wherein the apparatus has a unitary construction comprising a single piece of plastic.
